(19) United States
(12) Patent Application Publication
(10) Pub. No.: US 2025/0325565 A1
(43) Pub. Date: Oct. 23, 2025

(54) ERAVACYCLINE FOR TREATING CANCER

(71) Applicant: B. G. NEGEV TECHNOLOGIES AND APPLICATIONS LTD., Beer Sheva (IL)

(72) Inventors: Shimon BEN-SHABAT, Beer-Sheva (IL); Bracha SHAPIRA, Beer-Sheva (IL); Lior ROKACH, Beer-Sheva (IL); Guy SHTAR, Beer-Sheva (IL); Eyal MAZUZ, Beer-Sheva (IL); Adi JABARIN, Beer-Sheva (IL)

(73) Assignee: B. G. NEGEV TECHNOLOGIES AND APPLICATIONS LTD., Beer Sheva (IL)

(21) Appl. No.: 18/870,011

(22) PCT Filed: May 29, 2023
(86) PCT No.: PCT/IL2023/050548

§ 371 Date: Nov. 27, 2024

Related U.S. Application Data
(60) Provisional application No. 63/346,868, filed on May 29, 2022.
(60) Provisional application No. 63/404,251, filed on Sep. 7, 2022.
(60) Provisional application No. 63/430,466, filed on Dec. 6, 2022.

(51) Int. Cl. A61K 31/65; A61K 45/06; A61P 35/00

(52) U.S. Cl. CPC A61K 31/65; A61K 45/06; A61P 35/00

(57) ABSTRACT

Methods of treating pancreatic cancer or metastasis thereof by administering eravacycline or a derivative thereof are provided. Pharmaceutical compositions including eravacycline or a derivative thereof for use in treating pancreatic cancer or a metastasis thereof are also provided.

### CROSS-REFERENCE TO RELATED APPLICATIONS

This application claims the benefit of priority of: U.S. Provisional Application No. 63/346,868, filed May 29, 2022, and U.S. Provisional Application No. 63/404,251, filed Sep. 7, 2022 both titled “IDENTIFICATION AND CHARACTERIZATION OF DRUGS WITH NOVEL ANTI-CANCER ACTIVITY, SELECTED BY COMPUTATIONAL DRUG REPURPOSING STUDY, USING ARTIFICIAL INTELLIGENCE (AI) DEEP LEARNING MODELS”, and U.S. Provisional Application No. 63/430,466, filed Dec. 6, 2022 titled “ERAVACYCLINE FOR TREATING CANCER”, all of which are hereby incorporated by reference in their entirety.

### FIELD OF INVENTION

The present invention is in the field of cancer treatment.

### BACKGROUND OF THE INVENTION

With minimal treatment options, pancreatic adenocarcinoma (PDAC) is a devastating disease. This cancer is recognized as one of the deadliest malignancies, and it is the leading cause of cancer-related deaths in Western countries. The presence of multiple changes in signaling pathways may explain some of this cancer's resistance mechanisms. Pancreatic cancer patients' survival rate is estimated to average five years at most. Chemotherapy, radiation, and surgery are widely used, but do not result in significant improvements in clinical outcomes. The lack of treatment options emphasizes the need for new approaches for treating and managing this deadly disease.

Repurposing drugs that have already been approved by the Food and Drug Administration (FDA) for other indications has become a widely accepted approach for discovering new anticancer drugs, reducing costs, and eliminating the need for toxicological tests. An existing drug may have a higher success rate than a potential drug in the FDA's new chemical entity (NCE) track. A deep learning approach for identifying and predicting new indications for existing drugs was extended to other areas. In the case of pancreatic cancer, where the mechanisms of the disease remain unclear, this approach may be extremely valuable. Drug reuse for PDAC has received increasing attention in recent years, however research in this area has mainly been driven by hypotheses based on the overlap between an existing pharmacological mechanism of action (MOA) and the causes of the disease. While some of the drugs proposed showed promising anticancer activity, few successes have been reported.

As drug databases have grown, machine learning (ML) based approaches for changing a drug's designation have emerged. These tools identify new drug-disease interactions. ML based approaches can then be optimized to repurpose a drug. Disadvantages of existing drug repurposing tools include the facts that they are usually not disease-specific, and they sometimes include data on drug mechanisms and pathways obtained from diverse biological frameworks which are not available for the relevant drugs. Tools that predict specific properties or activity based on the chemical structure may result in more accurate predictions. New modalities for treating cancer in general and PDAC in particular are greatly needed.

### SUMMARY OF THE INVENTION

The present invention, in some embodiments, provides methods of treating or preventing pancreatic cancer or a metastasis thereof by administering eravacycline or a derivative thereof to a subject in need thereof. Pharmaceutical compositions comprising eravacycline or a derivative thereof for use in treating pancreatic cancer or a metastasis thereof in a subject in need thereof are also provided.

According to a first aspect, there is provided a method of treating or preventing pancreatic cancer or a metastasis thereof in a subject in need thereof, the method comprising administering to the subject a therapeutically effective amount of eravacycline or a derivative thereof, thereby treating or preventing pancreatic cancer or a metastasis thereof.

According to another aspect, there is provided a method of treating or preventing a cancer comprising expression of a mutated p53 bearing a mutation wherein Tyrosine in position 220 is substituted by Cysteine (Y220C) in a subject in need thereof, the method comprising administering to the subject a therapeutically effective amount of eravacycline or a derivative thereof, thereby treating or preventing cancer comprising expression of a mutated p53 bearing a Y220C mutation in the subject.

According to another aspect, there is provided a pharmaceutical composition comprising a therapeutically effective amount of eravacycline or a derivative thereof for use in treating or preventing pancreatic cancer or a metastasis thereof in a subject in need thereof.

According to another aspect, there is provided a pharmaceutical composition comprising a therapeutically effective amount of eravacycline or a derivative thereof for use in treating or preventing a cancer comprising expression of a mutated p53 bearing a Y220C mutation in a subject in need thereof.

According to some embodiments, the cancer is pancreatic cancer or a metastasis thereof.

According to some embodiments, the eravacycline is represented by formula I:

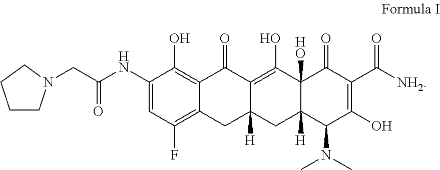 [SMILES: CN(C)[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12]

According to some embodiments, the eravacycline is a salt or crystalline form of eravacycline.

According to some embodiments, the eravacycline is eravacycline dihydrochloride.

According to some embodiments, the pancreatic cancer is pancreatic ductal adenocarcinoma (PDAC).

According to some embodiments, the administering is systemic administering.

According to some embodiments, the administering comprises administering a pharmaceutical composition comprising a therapeutically effective amount of eravacycline and a pharmaceutically acceptable carrier, excipient, or adjuvant.

According to some embodiments, the subject does not suffer from a bacterial infection treatable with eravacycline.

According to some embodiments, the treating comprises at least one of: increasing cancer cell apoptosis, increasing expression of cleaved poly(ADP-ribose) polymerase 1 (cPARP1) in a cancer cell, decreasing expression of DNA polymerase kappa (POLK) in a cancer cell, decreasing expression of mutated p53 in a cancer cell, and decreasing migration of a cancer cell, in the subject.

According to some embodiments, the method further comprises administering at least one other conventional cancer therapy.

According to some embodiments, the pharmaceutical composition is formulated for systemic administration.

According to some embodiments, the pharmaceutical composition further comprises a pharmaceutically acceptable carrier, excipient, or adjuvant.

According to some embodiments, the treating comprises at least one of: increasing cancer cell apoptosis, increasing expression of cPARP1 in a cancer cell, decreasing expression of POLK in a cancer cell and decreasing migration of a cancer cell, in the subject.

According to some embodiments, the pharmaceutical composition is further being used in combination with at least one other conventional cancer therapy.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 includes a cartoon showing an embodiment of a method of the invention for repurposing drugs with potential anticancer activity using machine learning: 1) A labeled dataset is compiled from DrugBank, cancer.com, ClinicalTrials.gov, and MeSH. 2) The dimensionality of drug-target interactions and drug-drug interactions is reduced; this information serves as tabular features. 3) A message passing neural network is trained to identify molecules with potential anticancer activity. The input consists of the molecule structure and tabular features. The model is used to rank all approved drugs by probability of anticancer activity. 4) The mechanism of action of the approved drugs is predicted using a message passing neural network trained on ExCAPE-DB which consists of 1.5 million molecules and 1,300 yeast targets. 5) A pharmacologist identifies promising candidates using both models' output. 6) The anticancer activity of the selected candidates is validated in vitro and in vivo.

FIGS. 2A-2F include graphs showing that Eravacycline suppresses cell growth and proliferation of pancreatic cancer cells. (2A-2C) Line graphs of the cytotoxic effects of (2A) omadacycline, (2B) tigecycline, and (2C) eravacycline in AsPC-1, BxPC-3, A-549, MCF-7, HT-29 cells for 72 h. (2D) Line graph of the tigecycline, eravacycline, and omadacycline anticancer activity in the BxPC-3 cell line. (2E) Line graph of inhibition rates of two human PDAC cell lines and one human normal pancreatic cell line (HPNE) treated with increasing concentrations of eravacycline for 72 h. (2F) Bar graph of inhibition rates of BxPC-3 cell line treated with increasing concentrations of eravacycline, doxorubicin, and gemcitabine for 72 h. Cell viability was determined by performing an XTT assay, which was performed to measure the IC50 values. At least three independent experiments were conducted. All data are shown as the mean±SD. A comparison between two groups was performed using a student's t-test, and comparisons between multiple groups were performed using a one-way ANOVA. ***P<0.001. ns, not significant. P-value <0.05 was considered statistically significant.

FIGS. 3A-3B include micrographs and a vertical bar graph showing that eravacycline inhibits cell migration in human PDAC cell lines. (3A) Micrographs of migration assessment evaluated by wound healing assay of BxPC-3 cells after cultured in medium only (control) or medium with 10 μM of eravacycline for the indicated time. Scale bar, 100 μm. (3B) Bar graph quantification of the effect of 10 μM of eravacycline on wound closure in BxPC-3 cells. At least three independent experiments were conducted. All data are shown as the mean±SD. A comparison between two groups was performed using a student's t-test, and comparisons between multiple groups were performed using a one-way ANOVA. **P<0.01, ***P<0.001. ns, not significant. P-value <0.05 was considered statistically significant.

FIGS. 4A-4D include plots, graphs, images, and tables, showing that eravacycline induces apoptosis in human pancreatic cancer cells. (4A-4B) Dot plots (4A) and bar graph quantification (4B) of apoptosis rates of BxPC-3 cells after exposed to control, 1 μM doxorubicin, 0.1 μM gemcitabine, 10 μM eravacycline or 25 μM eravacycline for 72 h by flow cytometry. Statistical analysis of apoptosis intensity in treated cells was performed in vitro. (4C-4D) BxPC-3 cells were treated with 1 μM doxorubicin, 0.01 μM gemcitabine, 0.1 μM gemcitabine or increasing concentrations of eravacycline for 72 h. (4C) The expression of cell apoptosis-related proteins, C-PARP1, was detected using Western blot assay; actin was used as control. At least three independent experiments were conducted. (4D) Bar graph quantification of C-PARP1 expression. All data are shown as the mean±SD. A comparison between two groups was performed using a student's t-test, and comparisons between multiple groups were performed using a one-way ANOVA. *P<0.05; **P<0.01, ***P<0.001. ns, not significant. P-value <0.05 was considered statistically significant.

FIGS. 5A-5I include images and graphs showing that eravacycline reduced POLK expression in BxPC-3 cells. (5A-5B) BxPC-3 cells were treated with increasing concentrations of eravacycline for 72 h. (5A) The expression of POLK-related protein was detected using Western blot assay; actin was used as control. (5B) Bar graph of quantification of POLK protein expression. (5C-5D) Western blot (5C) of POLK protein baseline expression levels in BxPC-3 and HPNE cells. At least three independent experiments were conducted. (5D) Bar graph quantification of POLK protein expression. All data are shown as the mean±SD. A comparison between two groups was performed using a student's t-test, and comparisons between multiple groups were performed using a one-way ANOVA. *P<0.05; ***P<0.001. ns, not significant. P-value <0.05 was considered statistically significant. (5E) Western blot of p53 protein expression after treatment with various concentrations of eravacycline, gemcitabine and doxorubicin. Upper band is p53. Lower band is actin. (5F) Bar graph quantification of the protein expression shown in 5E. (5G) Western blot of p53 protein expression in pancreatic cancer cell lines BxPC-3 and Panc-1scr and healthy pancreatic cell lines HPNE and Panc1-p53ko. (5H) Bar graph quantification of the protein expression shown in 5G. (5I) Line graph of survival of various pancreatic cells after culture with eravacycline.

FIGS. 6A-6D include graphs and images showing that eravacycline inhibits tumor growth in xenograft model of human PDAC cells in vivo. (6A) Line graph of mouse weight averages for each group. (6B) Photographs of tumors resected from mice in each group. (6C) Line graph of the percentage of change in the xenograft tumor size (volume) in the mice groups: the control group, the gemcitabine group (25 mg/kg), and the eravacycline group (10 mg/kg). (6D) Bar graph of tumor weight average for each group. At least three independent experiments were conducted. All data are shown as the mean±SD. A comparison between two groups was performed using a student's t-test, and comparisons between multiple groups were performed using a one-way ANOVA. *P<0.05; ***P<0.001. P-value <0.05 was considered statistically significant.

### DETAILED DESCRIPTION OF THE INVENTION

The present invention, in some embodiments, provides methods of treating or preventing cancer in a subject in need thereof by administering eravacycline to the subject. The present invention further concerns a composition comprising eravacycline for use in treating cancer.

Based on the recent success in identifying Halicin, a new antibiotic, using ML (see Stokes et al., “A deep learning approach to antibiotic discovery”, Cell, 2020 Feb. 20; 180(4):688-702.e131, herein incorporated by reference in its entirety), herein the same message passing neural network (Chemprop) was trained to predict the anticancer activity of small molecules, using drug data collected from various sources (e.g., DrugBank, clinicaltrials.gov). In addition to analyzing the chemical structure of the drugs, this anticancer prediction model was extended to consider drug-target interaction (DTI) and drug-drug interaction (DDI) information and the resulting improvement in the model's accuracy was demonstrated. This model was used to predict the anticancer activity of chemical structures and drugs that have not been tested in clinical trials for cancer; specifically, the model was used to predict the anticancer activity of all FDA-approved molecules as a means of identifying approved molecules with unknown anticancer potential. To explain the MOA behind the predicted anticancer drugs and complete the virtual screening process, an in-silico yeast screening ML model weas developed based on an extensive database with over 1.5 million molecules. This model was used to predict three possible outcomes (active, inactive, and no relation) for over 1,300 targets.

By analyzing the two models' (i.e., the ML anticancer prediction model and the in-silico yeast screening ML model) predictions for the set of FDA-approved drugs, three antibacterial drugs from the tetracycline family were found to have high anticancer activity scores: eravacycline, tigecycline, and omadacycline (listed in order of their ranking). While eravacycline and omadacycline have never been tested for their potential anticancer activity, tigecycline, which was approved as an antibiotic in 2005, demonstrated possible anticancer activity and, in the case of PDAC, exerted its action via downregulating of CCNE2. Eravacycline and omadacycline were developed and approved in 2018 and showed excellent antibacterial activity. Drugs in the tetracycline family are broad-spectrum antimicrobial agents widely used in human medicine. They are also used to treat a variety of diseases and disorders, including cancer and inflammation.

By a first aspect, there is provided a method of treating or preventing cancer, the method comprising contacting a cell of the cancer with eravacycline, thereby treating the cancer.

By another aspect, there is provided a composition comprising eravacycline for use in treating or preventing cancer.

In some embodiments, the cancer is a solid cancer. In some embodiments, the cancer is a tumor. In some embodiments, the cancer is selected from hepato-biliary cancer, cervical cancer, urogenital cancer (e.g., urothelial cancer), testicular cancer, prostate cancer, thyroid cancer, ovarian cancer, nervous system cancer, ocular cancer, lung cancer, soft tissue cancer, bone cancer, pancreatic cancer, bladder cancer, skin cancer, intestinal cancer, hepatic cancer, rectal cancer, colorectal cancer, esophageal cancer, gastric cancer, gastroesophageal cancer, breast cancer (e.g., triple negative breast cancer), renal cancer (e.g., renal carcinoma), skin cancer, head and neck cancer, leukemia and lymphoma. In some embodiments, the cancer is pancreatic cancer. In some embodiments, the pancreatic cancer is pancreatic ductal adenocarcinoma (PDAC). In some embodiments, the cancer is a metastatic cancer. In some embodiments, the cancer is a metastasis. In some embodiments, the metastasis is a pancreatic cancer metastasis. In some embodiments, the cancer is pancreatic cancer or a metastasis thereof. In some embodiments, the cancer is not lung cancer. In some embodiments, the cancer is not breast cancer. In some embodiments, the cancer is not colon cancer. In some embodiments, the cancer is selected from pancreatic, esophageal, colorectal, head and neck and larynx cancer. In some embodiments, the cancer is selected from esophageal, colorectal, head and neck and larynx cancer.

In some embodiments, the cancer overexpresses POLK. In some embodiments, the method further comprises determining POLK expression in the cancer and administering eravacycline to a POLK overexpressing cancer. In some embodiments, overexpressing is as compared to a healthy cell or tissue. In some embodiments, the healthy cell or tissue is the same tissue or cell type as the cancer. In some embodiments, the healthy tissue or cell type is a pancreas or pancreatic cells. In some embodiments, overexpression comprises at least a 10, 15, 20, 25, 30, 35, 40, 45, 50, 55, 60, 65, 70, 75, 80, 85, 90, 95 or 100% increase over the healthy cell or tissue, or any value and range therebetween. Each possibility represents a separate embodiment of the invention. In some embodiments, overexpression is at least a 25% increase. In some embodiments, the cancer expresses mutant p53. In some embodiments, the cancer comprises at least one cell expressing mutant p53. In some embodiments, the mutation is a gain of function mutation. In some embodiments, the mutation is a tyrosine 220 to cysteine (Y220C) mutation. In some embodiments, the mutation is a mutation equivalent to the Y220C mutation which causes the same gain of function. In some embodiments, the mutation is not arginine 273 to histidine (R273H) or an equivalent mutation which causes the same gain of function. In some embodiments, mutant p53 is oncogenic p53. Cancers harboring p53 mutations are well known in the art and can be found in every tissue type, however, certain cancers show a particularly high incidence of such mutations. These include, for example, pancreatic, esophageal, colorectal, head and neck and larynx cancer. In some embodiments, the method further comprises determining mutant p53 expression in the cancer and administering eravacycline to a cancer expressing mutant p53. In some embodiments, the method further comprises determining the mutation status of p53 in the cancer and administering eravacycline to a cancer expressing mutant p53. Methods of measuring expression levels in cancer are well known in the art and any such method may be used. This includes for example PCR, western blotting, and sequencing to name but a few. In some embodiments, the method comprises receiving a sample from the subject comprising a cancer cell. In some embodiments, POLK expression is expression in the sample. In some embodiments, mutant p53 is mutant p53 in the sample.

Eravacycline is a halogenated tetracycline derivative with a well-known antibacterial function. The chemical structure of tetracyclines is formed by a linear fused tetracyclic ring (A-D), to which a variety of pharmacophores are attached. Structural modifications at the C7 and C9 positions of the tetracyclines' D ring have been considered the most promising approaches for enhancing the antibacterial activity that led to the discovery of tigecycline, omadacycline, and eravacycline. These drugs include a unique tail extension at the C9 position. The similarity in the tail structure of eravacycline and tigecycline is high compared to the similarity in the tail structure of eravacycline and omadacycline. In eravacycline and tigecycline, the tail at the C9 position is nearly identical and includes acidic hydrogen on the amide nitrogen and a lipophilic tail extension. The lack of the hydroxyl group at the C6 position results in greater lipid solubility in all three molecules. The essential difference between the structure of eravacycline and that of tigecycline and omadacycline is the fluorine atom in the C7 position. Eravacycline is a new tetracyclic analog with a fluorine atom at the C7 position and a pyrrolidinoacetamido group at the C9 position of the D ring.

Eravacycline is also known as (4S,4aS,5aR,12aS)-4-(Dimethylamino)-7-fluoro-3,10,12,12a-tetrahydroxy-1,11-dioxo-9-[2-(pyrrolidin-1-yl)acetamido]-1,4,4a,5,5a,6,11,12a-octahydrotetracene-2-carboxamide. It is available commercially as Xerava. It is provided in CAS number 1207283-85-9 and has the chemical formula C₂₇H₃₁FN₄O₈. In some embodiments, eravacycline is represented by formula I:

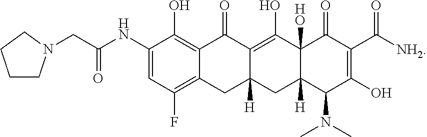 [SMILES: CN(C)[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12]

In some embodiments, eravacycline is a salt of eravacycline. In some embodiments, eravacycline is eravacycline dihydrochloride. Eravacycline dihydrochloride is provided in CAS number 1334714-66-7. And has the chemical formula C₂₇H₃₃CL₂FN₄O₈. In some embodiments, eravacycline is crystalline eravacycline. Crystalline forms of eravacycline are provided in International Patent Application WO2018/075767, which is hereby incorporated by reference in its entirety.

In some embodiments, eravacycline is a derivative of eravacycline. In some embodiments, the derivative comprises a fluorine atom in the C7 position of the D ring. In some embodiments, the derivative comprises a pyrrolidinoacetamido group at the C9 position of the D ring. The term “derivative”, as used herein, refers to a therapeutic compound based off eravacycline which retains anticancer function. In some embodiments, a derivative is synthesized from eravacycline.

As used herein, the terms “treatment” or “treating” of a disease, disorder, or condition (e.g., cancer) encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life.

In some embodiments, treating or preventing is treating. In some embodiments, treating or preventing is preventing. In some embodiments, treating comprises increasing cancer cell apoptosis. In some embodiments, treating comprises increasing cancer cell death. In some embodiments, treating comprises decreasing tumor size. In some embodiments, tumor size is tumor volume. In some embodiments, tumor size is tumor weight. In some embodiments, decreasing comprises a statistically significant change. In some embodiments, increasing comprises a statistically significant change. In some embodiments, a statistically significant change is at least a 10, 15, 20, 25, 30, 35, 40, 45, 50, 55, 60, 65, 70, 75, 80, 85, 90, 92, 95, 97, 99, 100, 150, 200, 250, 300, 350, 400, 450 or 500% change, or any value and range therebetween. Each possibility represents a separate embodiment of the invention. In some embodiments, the effect of eravacycline on cancer cell apoptosis/death and/or tumor size is superior to the effect produced by tigecycline. In some embodiments, the effect of eravacycline on cancer cell apoptosis/death and/or tumor size is superior to the effect produced by doxorubicin. In some embodiments, the effect of eravacycline on cancer cell apoptosis/death and/or tumor size is superior to the effect produced by gemcitabine. In some embodiments, the effect of eravacycline on cancer cell apoptosis/death and/or tumor size is superior to the effect produced by omadacycline.

In some embodiments, treating comprises increasing expression of cleaved poly(ADP-ribose) polymerase 1 (cPARP1) in a cancer cell. In some embodiments, treating comprises increasing expression of cPARP1 in the tumor. In some embodiments, expression is protein expression. Cleaved PARP1 is an indicator of cell death and so an increase indicates an increase in death of the cancer cells. Methods of measuring cPARP1 expression levels are well known in the art and disclosed hereinbelow; any such method can be used.

In some embodiments, treating comprises decreasing expression of DNA polymerase kappa (POLK) in a cancer cell. In some embodiments, treating comprises decreasing expression of POLK in the tumor. In some embodiments, expression is protein expression. In some embodiments, expression is mRNA expression. In some embodiments, decreasing comprises decreasing replication of a cancer cell. In some embodiments, replication is DNA replication. In some embodiments, replication is cell division. Methods of measuring POLK are well known in the art and disclosed hereinbelow; any such method can be used.

In some embodiments, treating comprises decreasing migration of a cancer cell. In some embodiments, decreasing migration comprises decreasing metastasis. In some embodiments, decreasing metastasis comprises decreasing the rate of metastasis. In some embodiments, decreasing metastasis comprises decreasing the number of metastases. In some embodiments, decreasing metastasis comprises decreasing the rate of metastasis and decreasing the number of metastases. Method of measuring migration are well known in the art and disclosed hereinbelow; any such method can be used.

In some embodiments, contacting the cancer cell with eravacycline comprises administering eravacycline to the subject. In some embodiments, treating cancer comprises administering eravacycline to the subject. In some embodiments, the subject is a mammal. In some embodiments, the subject is a human. In some embodiments, the subject suffers from or is afflicted with cancer. In some embodiments, the subject is a subject in need of treatment. In some embodiments, the subject is a subject in need of cancer treatment. In some embodiments, the subject does not suffer from an infection. In some embodiments, the infection is a bacterial infection. In some embodiments, the infection is an infection treatable with eravacycline. Eravacycline is approved for the treatment of complicated urinary tract infection (cUTI) and complicated intra-abdominal infection (cIAI) due to multidrug-resistant Gram-positive, Gram-negative and anaerobic bacteria. In some embodiments, the subject does not suffer from a urinary tract infection or an intra-abdominal infection.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of eravacycline to a patient in need thereof. Other suitable routes of administration can include parenteral, subcutaneous, oral, intramuscular, intratumoral or intraperitoneal. Oral and intravenous formulations of eravacycline are commercially available and can be used in the method of the invention. In some embodiments, administering is systemic administration. In some embodiments, administering is intravenous administration. In some embodiments, administering is oral administration. In some embodiments, administering is intratumoral administration.

The dosage administered will be dependent upon the age, health, and weight of the recipient, kind of concurrent treatment, if any, frequency of treatment, and the nature of the effect desired.

In some embodiments, the eravacycline is therapeutically effective amount of eravacycline. In some embodiments, a therapeutically effective amount is a therapeutically effective dose. The term “therapeutically effective amount” refers to an amount of a drug effective to treat a disease or disorder (e.g., cancer) in a mammal. The term “a therapeutically effective amount” refers to an amount effective, at dosages and for periods of time necessary, to achieve the desired therapeutic or prophylactic result (e.g., treating cancer). The exact dosage form and regimen would be determined by the physician according to the patient's condition. In some embodiments, the dosage is the dosage used to treat a bacterial infection. In some embodiments, the dose is equivalent to about 10 mg/kg body weight in mice. In some embodiments, the dose is about 0.8 mg/kg body weight. In some embodiments, the dose is about 1 mg/kg body weight. In some embodiments, the dose is about 1.5 mg/kg body weight.

In some embodiments, administering comprises administering a composition comprising eravacycline. In some embodiments, the composition is a pharmaceutical composition. In some embodiments, the composition comprises a therapeutically effective amount of eravacycline. In some embodiments, the composition is formulated for administration to a subject. In some embodiments, the composition is formulated for systemic administration. In some embodiments, the composition is formulated for oral administration. In some embodiments, the composition is formulated for intravenous administration. In some embodiments, the composition is formulated for intratumoral administration.

In some embodiments, the composition further comprises a pharmaceutically acceptable carrier, excipient, or adjuvant. As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers, and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers, and the like. Liposomes for use with the presently described peptides are formed from standard vesicle-forming lipids which generally include neutral and negatively charged phospholipids and sterol(s), such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein. In some embodiments, the composition consists of eravacycline. In some embodiments, the composition consists essentially of eravacycline. In some embodiments, the composition comprises eravacycline as the only therapeutic agent. In some embodiments, the therapeutic agent is a therapeutic anticancer agent. In some embodiments, the composition is devoid of another therapeutic agent other than eravacycline. In some embodiments, the eravacycline is essentially pure eravacycline. In some embodiments, the composition comprises an active agent and a carrier, wherein the active agent consists essentially of eravacycline.

As used herein, the term “consists or consisting essentially of” denotes that a given compound or substance constitutes the vast majority of the active ingredient's portion or fraction of the composition.

In some embodiments, consists essentially of means that eravacycline constitutes at least 95%, at least 98%, at least 99%, or at least 99.9% by any one of: weight, mole, or molarity, of the composition, or any value and range therebetween. Each possibility represents a separate embodiment of the invention.

In some embodiments, the subject is administered eravacycline as a monotherapy. In some embodiments, the subject is administered eravacycline as part of a combination therapy. In some embodiments, the method further comprises administering at least one other cancer therapy. In some embodiments, the other cancer therapy comprises conventional cancer therapy. Conventional cancer therapies are well known in the art and include, but are not limited to, for example, chemotherapy, immunotherapy, radiation therapy and targeted therapy. Any such cancer therapy may be combined with eravacycline as part of a combination therapy. In some embodiments, the composition comprising eravacycline, is for use in combination with another cancer therapy.

As used herein, the term “about” when combined with a value refers to plus and minus 10% of the reference value. For example, a length of about 1,000 nanometers (nm) refers to a length of 1000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

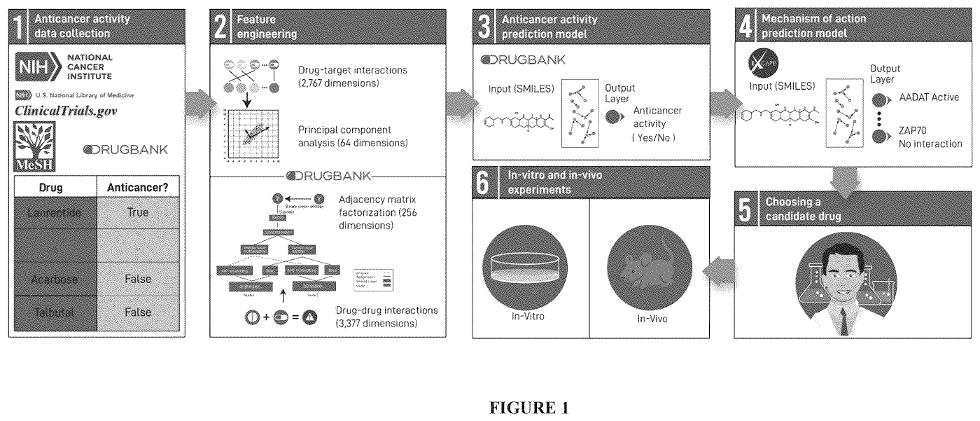

An overview of the methodology used herein is presented in FIG. 1. Anticancer training data was collected from various sources (FIG. 1.1). Then, a feature engineering process was performed (FIG. 1.2) and a deep neural network was trained to rank drugs with anticancer potential (FIG. 1.3); another model was used to predict the MOAs by performing in-silico yeast screening (FIG. 1.4). Finally, candidates were selected by a pharmacologist (FIG. 1.5) and validated in in vitro and in vivo experiments (FIG. 1.6). The methodology's phases are described in detail in the subsections that follow.

### Anticancer Activity Data Collection

To train the anticancer activity prediction model, a set of positive drugs consisting of 451 molecules with known anticancer activity were collected from DrugBank 5.1.8, ClinicalTrials.gov, and cancer.com, along with the drugs' associated Medical Subject Headings (MeSH) terms (FIG. 1.1). Eight hundred negative drugs without anticancer activity were randomly sampled from the list of FDA approved drugs after filtering out drugs that were already examined in cancer-related clinical trials (as reported in ClinicalTrials.gov) or drugs that are chemically similar to those drugs. The chemical similarity measure from the DrugBank website (drugbank.ca) was utilized. Afterwards, a pharmacologist manually validated the list of positive and negative drugs collected.

### Training Anticancer Activity Prediction Model

The set of drugs collected was then used to train a directed message passing neural network (Chemprop) model to predict the molecules' anticancer potential (FIG. 1.3). The model was trained to solve a binary classification problem. When predicting, the model assigns a continuous score quantifying the certainty of the prediction for each molecule. The Chemprop program is available at github.com/swansonk14/chemprop.

The model was evaluated using a balanced scaffold-based split, which was repeated three times. An ensemble of two Chemprop models was used. The contribution of additional drug features was also assessed (FIG. 1.2): (1) Drug target interaction (DTI) information, which was obtained from DrugBank 5.1.8 and compressed, using principal component analysis (PCA), from a binary matrix of 2,767 targets and 5,857 drugs to a 64-dimensional representation of the drugs; and (2) Drug-drug interaction (DDI) information (also obtained from DrugBank 5.1.8), originally represented by a rectangular binary matrix of 3,377 drugs and reduced to a 256-dimensional compressed representation using AMFP, which performs factorization-like compression on the matrix. Missing information was filled in based on the chemically most similar single drug; similarity was calculated as described in the previous subsection. For molecules that have no similar molecules using DrugBank's default threshold of 0.7, the average DTI and DDI information was used. The area under the receiver operating characteristic curve (AUC) served as the primary evaluation metric for the proposed models, but the results were also present based on the area under the precision-recall curve (AUPR) metric.

### In-Silico Yeast Screening

A second message passing neural network was also trained for in-silico yeast screening (see Ojima et al., “Use of fluorine in the medicinal chemistry and chemical biology of bioactive compounds—a case study on fluorinated taxane anticancer agents”, Chembiochem 5, 628-635, herein incorporated by reference) which can explain the MOA of drugs. The ExCAPE-DB20 database, which includes over 1.5 million molecules, 1,300 target yeast proteins, and 70 million DTIs (FIG. 1.4), was used to train the model. The model's output consists of one outcome (active, inactive, and no relation) for each target. Due to the size of the dataset and computational limitations, the model was evaluated by performing a single scaffold-based train-validation-test split of 70%, 10%, and 20%, respectively.

Inspired by test-time augmentation techniques, which are beneficial for creating accurate models, the weighted average prediction of similar drugs was used. Here, a drug from the tetracycline family was focused on (FIG. 1.5). Hence, the predictions of seven antibiotics from the tetracycline family who have known anticancer activity was used. The weights were based on the chemical similarity of the drugs. To set the classification thresholds for each target, the prediction score of the nth ranked sample was calculated, where n is the number of positive samples of a given target in the test set.

### Identifying Repurposable Drugs

The trained anticancer model was used to predict repurposable drugs with potential anticancer activity. Each approved drug found in DrugBank 5.1.8 was assigned an anticancer activity score by the trained anticancer model, and the drugs were ranked according to the score. The ranked list was analyzed, and commercially available drugs were given higher priority for further examination (FIG. 1.5). A few drugs such as trofosfamide (ranked 15th) and Fenretinide (ranked 34th) were found to have some known anticancer activity mentioned in the literature; such drugs were added to the training set, and the model was retrained. As such, those drugs did not continue to the in vitro experiment. After considering the anticancer potential and commercial availability, eravacycline (ranked 17th of the 1,352 approved molecules) was selected for further investigation, since, based on the review of the literature, it has not yet been examined for oncological use.

### Cell Culturing

Human PDAC (AsPC-1, BxPC-3, Panc-1 scr, Panc-1 p53-R273H knock-out (KO) hTERT-HPNE), breast (MCF-7), lung (A549), and colon (HT-29) cancer cell lines were purchased from the American Type Culture Collection (ATCC). The MCF-7, A549, Panc-1 scr, Panc-1 p53 KO and HT-29 cell lines were grown in Dulbecco's Modified Eagle Medium (DMEM; Biological Industries, Beit HaEmek (BI), Israel), and the BxPC-3 and AsPC-1 cell lines were cultured in Roswell Park Memorial Institute-1640 (RPMI-1640; BI, Israel) medium. All media was supplemented with 10% fetal bovine serum (FBS), 200 μM L-glutamine (BI), and 1% penicillin-streptomycin (BI). Cells were cultured at 37° C. in a 5% CO2 humidified incubator.

### Tetracycline Derivative Treatment

Eravacycline dihydrochloride (MedChem Express (MCE)) was dissolved in deuterium-depleted water (DDW). Tigecycline (MCE) and omadacycline (MCE) were dissolved in dimethyl sulphoxide (DMSO). All drugs were prepared as 100 mM stock solutions. All cell lines were treated with DMSO as a control (less than 0.1%) or tigecycline, omadacycline, or eravacycline (at the indicated concentrations) for 72 hours. Cell viability and proliferation were determined using the XTT Cell Proliferation Assay Kit (Promega).

### Cell Proliferation Assay

The XTT Cell Proliferation Assay Kit was used to determine cell viability and proliferation. Cells were seeded in 96-well plates at a density of 4,000 cells per well and allowed to attach overnight in a 5% CO2 incubator at 37° C. After overnight incubation, cells were treated with culture medium or in a medium containing tigecycline, omadacycline, or eravacycline at different concentrations (1, 2, 5, 10, 25, and 50 μM) for the indicated time period. Then, 50 μl XTT reaction solution (0.1 ml activation solution+5 ml XTT reagent solution) was added to each well, and the plate was incubated at 37° C. for two hours. After shaking the plate gently to distribute the dye in the wells, the absorbance was measured with a spectrophotometer (Bio-RadiMark Microplate Absorbance reader) at a wavelength of 450 nm and a reference wavelength of 655 nm.

### Determination of Half Maximal Inhibitory Concentration (IC50) of Eravacycline, Tigecycline, and Omadacycline

To evaluate the half maximal inhibitory concentration (IC50) of tetracycline derivatives on five different cell lines (MCF-7, A549, HT-29, BxPC-3, and AsPC-1), the cells were seeded and allowed to attach overnight. All cells were washed with phosphate buffered saline (PBS) and then cultured in fresh media containing the drugs tigecycline, omadacycline, or eravacycline at an increasing concentration (0-50 μM) for 72 h. Inhibition of cell proliferation was determined using the XTT Cell Proliferation Assay Kit.

### Cell Migration and Scratch Wound Healing Assay

BxPC-3 cells were cultured in six well plates to full confluence; then the monolayer of the cells was scratched using a 200 μl pipette tip. Subsequently, PBS was used to wash and remove floating and damaged cells, and medium alone or medium containing 10 μM eravacycline was added to the cells for further culture. Cell migration over the denuded area was observed and captured at the indicated times with a Nikon ECLIPSE Ts2 fluorescence microscope, equipped with DS-Fi3 camera. The wound closure rates were also measured at the indicated time points.

### Flow Cytometry Analysis

Cells were cultured in 6-well plates containing different concentrations of eravacycline, gemcitabine, or doxorubicin (medium alone was used as a control) for 72 h. Afterward, cells were collected by trypsinization for flow cytometry analysis. Cells were resuspended in 100 μl binding buffer, then stained with 5 μl propidium iodide (PI) and 5 μl Annexin V-FITC (Annexin V-FITC Apoptosis Detection Kit with PI-BioLegend) at room temperature for 20 min in the dark. Finally, the cells were collected and analyzed by FACS (Sony SP6800 Spectral cell analyzer), and the data was analyzed using SP6800 spectral analyzer software.

Protein Extraction from Bxpc-3 Cells

Following the three-day incubation of BxPC-3 cells with medium alone as a control or medium containing different concentrations of eravacycline, gemcitabine, or doxorubicin, BxPC-3 cells were harvested and lysed. BxPC-3 cells were briefly lysed with a buffer containing 20 mM HEPES (pH 7.4), 150 mM NaCl, 1 mM EGTA, 1 mM EDTA, 10% glycerol, 1 mM MgCl2, 1% Triton X-100, and 10 μL of protease phosphatase inhibitors per 1 mL buffer. After incubation on ice for 45 min, the lysates were sonicated for 15 min (40% amplitude without pulses) and centrifuged at 12,000×g at 4° C., and the supernatants were collected. The supernatants were analyzed for protein concentration using the Bradford assay. Absorbance was recorded on a microplate reader at 595 nm (Bio-RadiMark Microplate Absorbance reader).

### Western Blot Analysis

To evaluate the expression of cell apoptosis-related proteins, cleaved Poly (ADP-ribose) polymerase 1 (C-PARP1) was detected using western blot assays. Actin was used as a control. Following the three days incubation of BxPC-3 cells with medium alone as a control or medium containing different concentrations of eravacycline, gemcitabine, or doxorubicin, cells were harvested and lysed. Then, 20 μg protein of each sample was extracted and mixed with Laemmli sample buffer (Bio-Rad, Hercules, CA, USA) containing 0.1% β-mercaptoethanol, boiled for 5 min at 95° C., and separated on a 10% SDS-PAGE. Proteins were transferred to a nitrocellulose membrane, blocked in 5% BSA with 0.5% Tween 20, and then probed overnight with primary antibodies to detect and analyze the expression levels of C-PARP1. Membranes were incubated for 1 h at room temperature with anti-rabbit secondary antibodies (1:1,000, Sigma-Aldrich). β-actin levels were determined using anti-β-actin (1:10,000, Sigma-Aldrich). Immune complexes were detected with a chemiluminescence reagent (Thermo Fisher Scientific, Pierce ECL Plus Substrate), followed by exposure to Kodak X-ray film (Rochester, NY, USA). Semi-quantitative analysis was performed for all Western blot experiments using a computerized image analysis system, (Mac Biophotonics ImageJ software version 1.53k14).

### Preparation and Processing of BxPC-3 Cell Xenograft Tumor Model

Six-week-old female athymic nude mice were purchased from Envigo (Jerusalem, Israel) and housed in pathogen-free conditions and fed a standard sterilized diet. The mice were housed in humidity- and temperature-controlled conditions, and the light/dark cycle was set at 12 h intervals. The animal protocol was reviewed and approved by the Committee for the Ethical Care and Use of Animals in Research at Ben-Gurion University of the Negev, complying with the Israeli Animal Welfare Law. BxPC-3 cells (4×10⁶) were injected subcutaneously into one flank of the mice. Tumor size was measured every other day based on caliper measurements of tumor length and width (volume=tumor length×width²×0.5236). Ten days later, when tumors were palpable and reached volumes of 50-150 mm³, the mice bearing tumors were randomly assigned to control or eravacycline groups (n=6). Treatment was administered every two days using either phosphate buffered saline (PBS), eravacycline (10 mg/kg by i.p. injection) or gemcitabine (25 mg/kg by i.p. injection) for 10 days. The tumor growth and mouse weight were measured every other day during the treatment period. After the last treatment, the mice were monitored for an additional six days, and then the experiment was terminated. The mice were then sacrificed, the tumors were excised, photographed, and measured, and the tumor tissues were snap-frozen, pending analysis.

### Example 1

### In-Silico Candidate Identification

Table 1 presents the performance of the anticancer prediction model used in this research. An AUC of 0.83 was achieved for a model that only considers the drug's molecular structure, however the best performing model was also trained using the drugs' DTI and DDI information, and the enhanced model obtained an AUC of 0.909. Eravacycline was ranked 17^{th} out of the 1,371 ranked molecules. This drug was given higher priority due to the known anticancer activity of tetracyclines and its commercial availability. The PCA processes decreased the number of dimensions from 2,767 to 64 while retaining 45% of the variance.

Table 1: Area under the receiver operating characteristic curve (AUC) and area under the precision-recall curve (AUPR) of a message passing neural network trained on the molecular structure of the drugs for different combinations of additional drug features. RDKIT 2D features are calculated by Chemprop using functionality found in RDKIT software.

TABLE 1

| Additional DRUG Features | AUC | AUPR |
|---|---|---|
| None | 0.806 | 0.703 |
| RDkit 2d | 0.827 | 0.722 |
| Drug-target interactions (DTI) | 0.853 | 0.772 |
| Drug-drug interactions (DDI) | 0.897 | 0.829 |
| DDI + DTI | 0.909 | 0.849 |

### Example 2

### In-Silico Yeast Screening

For the in-silico yeast screening model, an average AUC of 0.939 and AUPR of 0.99 was achieved for the task of identifying the absence of an interaction (note that the inverse of this class attribute can be used to identify the presence of any type of interaction); an AUC of 0.913 and an AUPR of 0.178 was achieved for identifying activation of a target; and an AUC of 0.95 and AUPR of 0.19 was achieved for identifying inactivation of a target. On average, 3.6% of the potential interactions in the test set exist. To identify candidate target proteins that can explain eravacycline's predicted anticancer activity, the first 30 predictions for eravacycline were manually analyzed, comparing each prediction with the threshold of each target; the threshold is the nth prediction score, where n is proportional to the number of positive cases in the training set (eravacycline was not part of the training set). The first predicted interaction for eravacycline is with CYP3A4; this prediction was confirmed by looking up the drug entry on the DrugBank website. Additional targets were identified by analyzing the model's output for eravacycline and targets that are known to take part in anticancer MOAs were focused on. The targets are P53, ALOX12, POLK, and BLM. The only target that was not associated with PDAC is POLK.

### Example 3

### Effect of Tetracycline Derivatives on Human Breast, Lung, Colon, and PDAC Carcinoma Cell Viabilities

To investigate the effect of tetracycline derivatives on cell proliferation in human breast, lung, colon, and PDAC cells, the inventors treated all cell lines (MCF-7, A549, HT-29, BxPC-3, and AsPC-1) with different concentrations of tigecycline, omadacycline, and eravacycline (1, 2, 5, 10, 25, and 50 μM), with medium used as a control, for 72 h. Then, an XTT assay was performed to determine the growth inhibition rate.

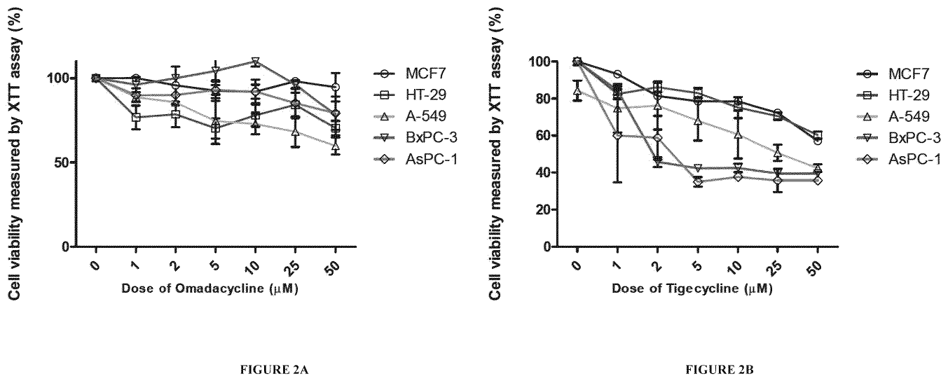

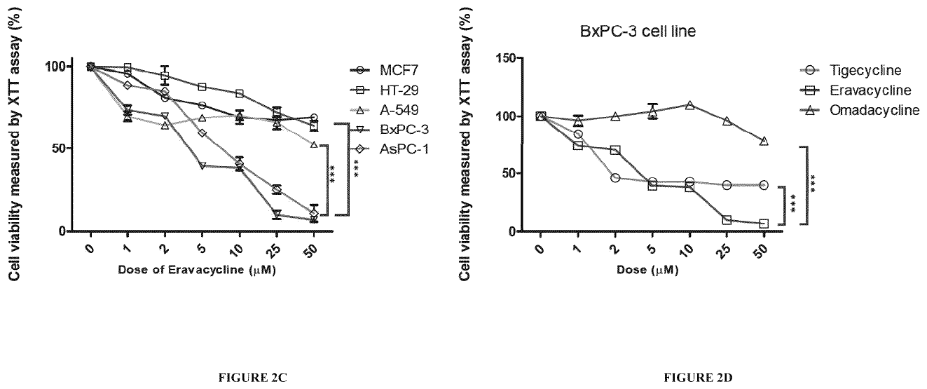

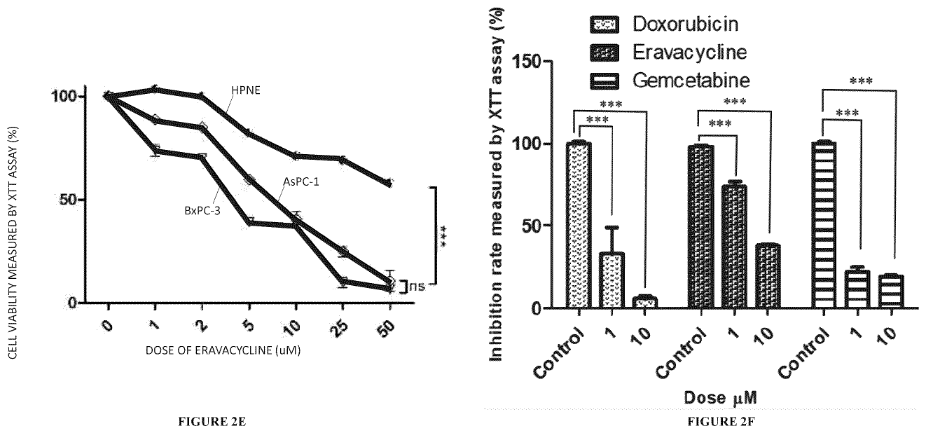

The results show that treatment with increasing concentrations of omadacycline had no significant effect on cell survival (FIG. 2A). The treatment of all cell lines with tigecycline for 72 h suppressed the proliferation of human PDAC (BxPC-3, AsPC-1) and A549 cell lines moderately in a concentration-dependent manner by up to 60-65%, while no significant effect was observed in the MCF-7 and HT-29 cell lines (FIG. 2B). Treatment of the cells with eravacycline for 72 h suppressed the proliferation of human PDAC cells (BxPC-3, AsPC-1) in a concentration-dependent manner by up to 90-93% (FIG. 2C). When administered at a concentration of 25 μM eravacycline was able to reduce the human BxPC-3 cells' viability more effectively than 25 μM of tigecycline (a 93% vs 60% reduction, respectively, where p=0.001). No significant effect was observed for eravacycline in the MCF-7, A549, and HT-29 cell lines (FIG. 2C). These results indicate that both PDAC cell lines had a significant reduction in cell survival in a dose-dependent manner following treatment with eravacycline (FIG. 2C). The IC50 of eravacycline on the BxPC-3 and AsPC-1 cells was 3.57 and 7.07 μM, respectively (FIG. 2C). In contrast, tigecycline needed 9.9 μM and 4.58 μM to inhibit 50% of the BxPC-3 and AsPC-1 cells, respectively, at the same time point (FIG. 2B).

After 72 hours of treatment, eravacycline was shown to significantly decrease the BxPC3-3 cells' growth by 90% (P<0.01) and 93% (P<0.001) at 25 and 50 μM concentrations, respectively (FIG. 2C), whereas it caused a 75% (P<0.05) and 89% (P<0.001) decrease in AsPC-1 cell growth at the same concentrations (FIG. 2C). It was thus clear that eravacycline was superior to both omadacycline and tigecycline for killing PDAC cells (FIG. 2D). The BxPC-3 cell line, which was relatively more sensitive to eravacycline, was selected for further study.

To compare the effect of eravacycline on pancreatic cancer cells to its effect on normal cells, the effect of eravacycline on cell proliferation was examined in one human normal pancreatic cell (HPNE cell line). The results revealed that HPNE cells were the most insensitive cells to eravacycline, showing significantly less inhibition than the other PDAC cell lines (FIG. 2E). To compare eravacycline's activity against BxPC-3 cancer cells to that of chemotherapeutic agents, the inventors examined the anticancer activity of doxorubicin and gemcitabine. After 72 h of treatment, doxorubicin significantly decreased BxPC-3 cells' growth by 67% (P<0.001) and 94% (P<0.001) at 1 and 10 μM concentrations, respectively (FIG. 2F). The percentage of BxPC-3 cell growth inhibition by gemcitabine was 77% and 80% at 1 and 10 μM, respectively (FIG. 2F). Eravacycline decreased BxPC-3 cells' growth by 27% and 63% at 1 and 10 μM concentrations, respectively (FIG. 2F).

These results indicate that eravacycline significantly inhibited cell proliferation of BxPC-3 cells dose-dependently, and it was more cytotoxic to PDAC cells than tigecycline or omadacycline (FIG. 2D). The results also show that the BxPC-3 cell line was the most sensitive to eravacycline, which was relatively less effective in breast (MCF-7), lung (A549), and colon (HT-29) cancer cell lines and in healthy pancreatic cells (FIG. 2E).

### Example 4

### Effect of Eravacycline on the Inhibition of Cell Migration in PDAC Cells

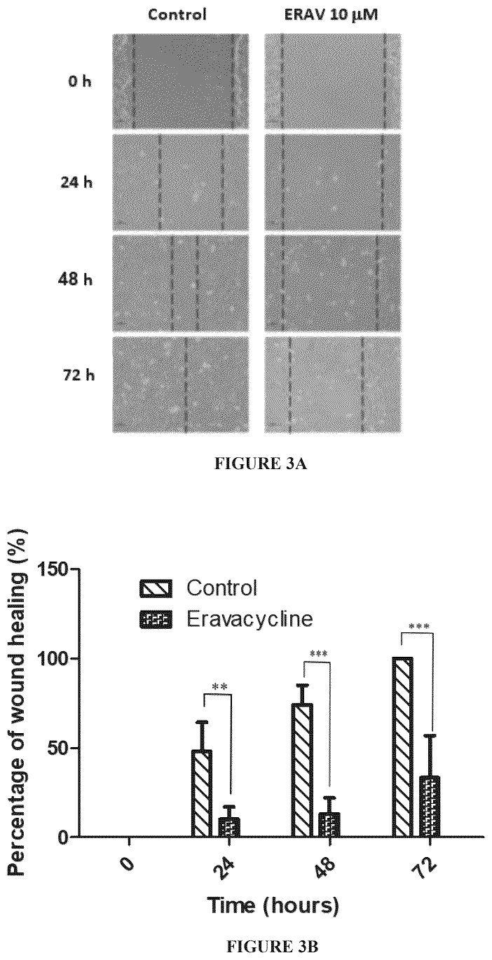

As one of the most malignant types of digestive tract cancer cells, PDAC cells have powerful migration and invasiveness capabilities. Therefore, the effect of eravacycline on cell migration abilities was investigated. The wound healing assay shows that BxPC-3 cells treated with 10 μM of eravacycline for 0, 24, 48, and 72 hours displayed a significantly lower migration rate to a wound introduced in a confluent monolayer of cells than that of the control group (FIGS. 3A-3B). These results indicate that eravacycline inhibited cell migration in human PDAC cells.

### Example 5

### Eravacycline's Effect on the Induction of Apoptosis in Pancreatic Cancer Cells

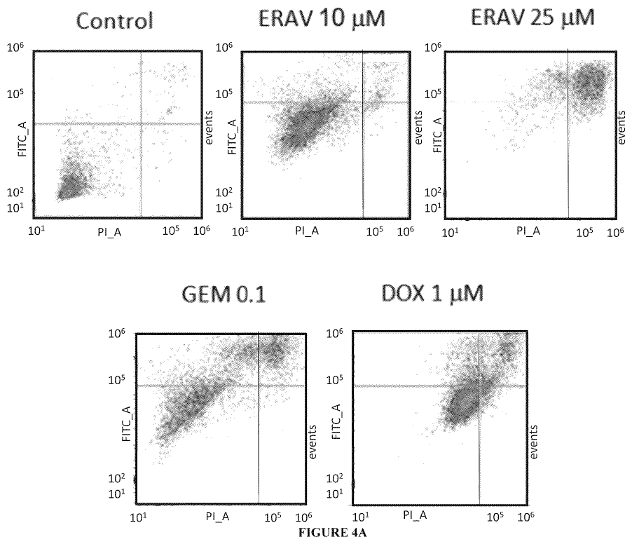

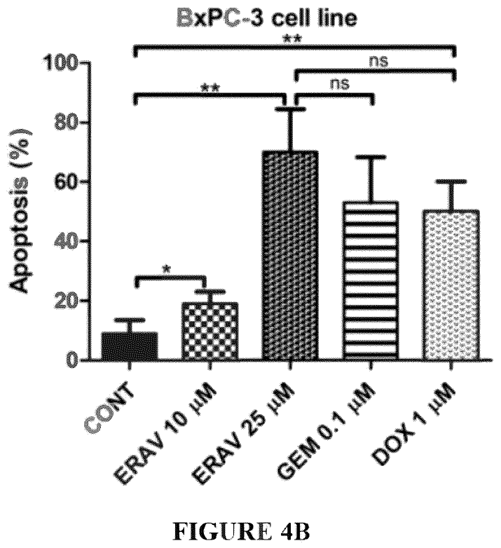

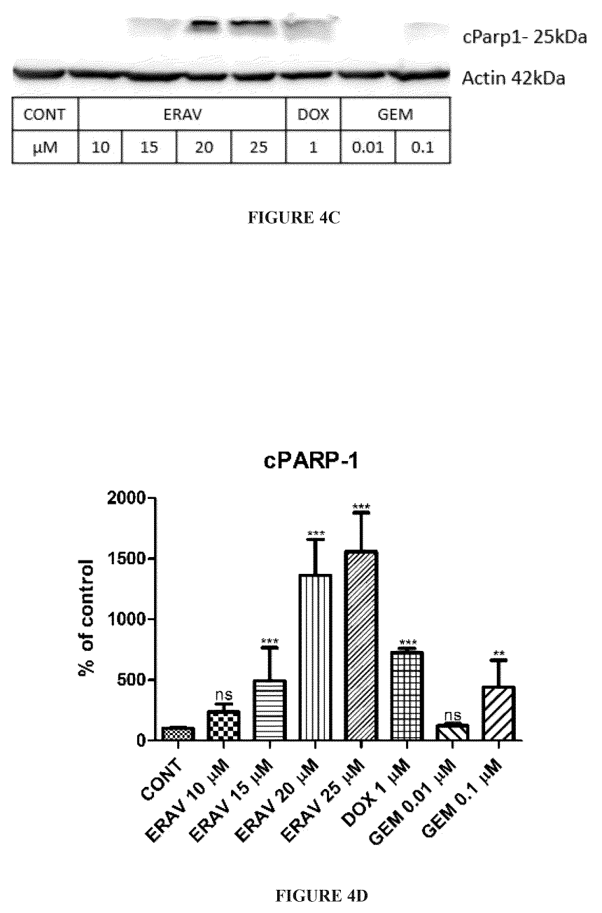

To examine eravacycline's effect on cell growth inhibition and the mechanism underlying this, the induction of apoptosis activated by eravacycline treatment in BxPC-3 cells was investigated by performing an Annexin V-FITC (V-fluorescein isothiocyanate) apoptosis assay. The effects of the treatment of BxPC-3 cells with increasing concentrations of eravacycline was evaluated after 72 h. A positive control of 1 μM of doxorubicin was evaluated at the same time point. BxPC-3 cells' treatment with 10 μM eravacycline resulted in a three-fold increase in the number of apoptotic cells (FIGS. 4A-4B). Twenty-five (25) μM eravacycline produced nearly 70% apoptosis which was superior to both gemcitabine (0.1 μM) and doxorubicin (1 μM) (FIG. 4B).

Western blot was used to evaluate the C-PARP1, and the results show that C-PARP1 increased significantly after treatment with 15 μM eravacycline compared to the control group (FIGS. 4C and 4D). These data indicate that eravacycline strongly influenced apoptosis in BxPC-3 cells and on a level equal to or greater than gemcitabine and doxorubicin.

### Example 6

### Eravacycline Suppresses the Survival of PDAC Cells by Inhibiting POLK and p53 Genes Expression

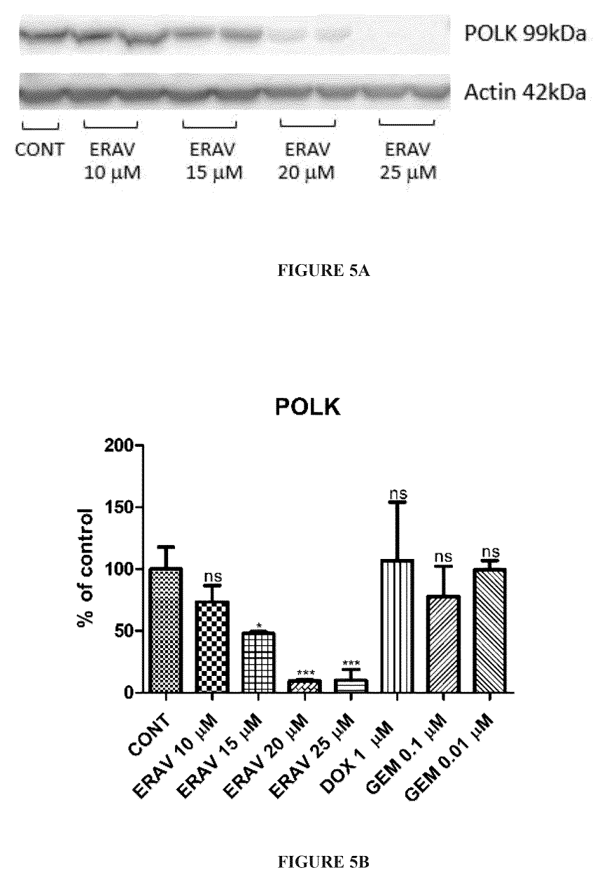

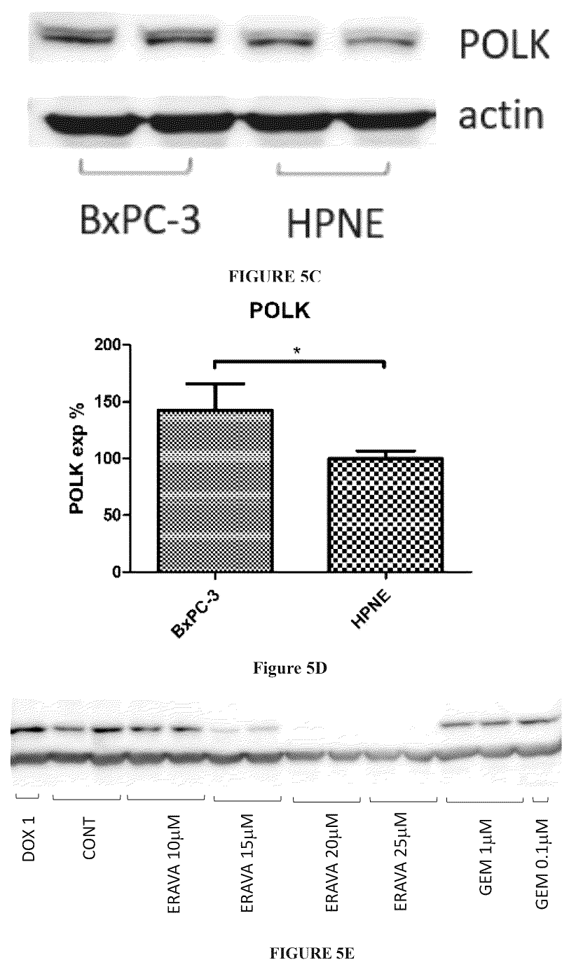

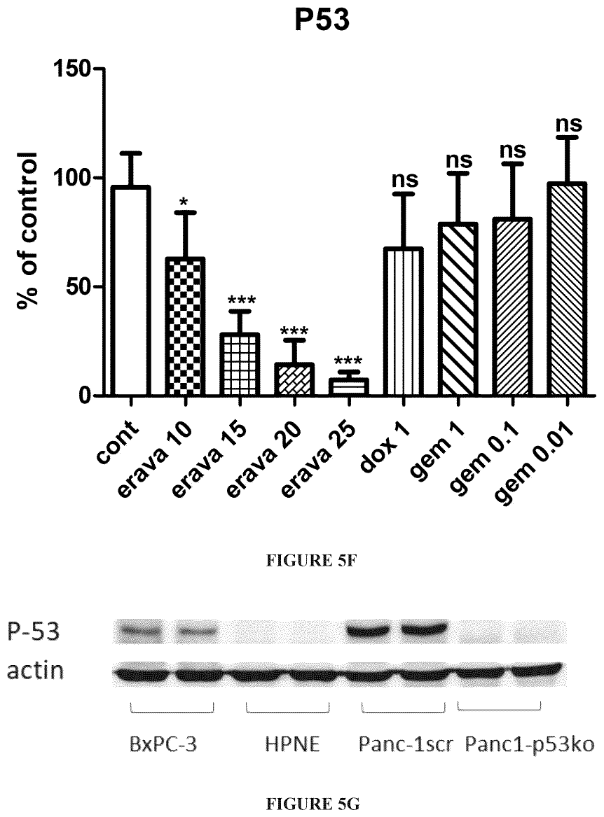

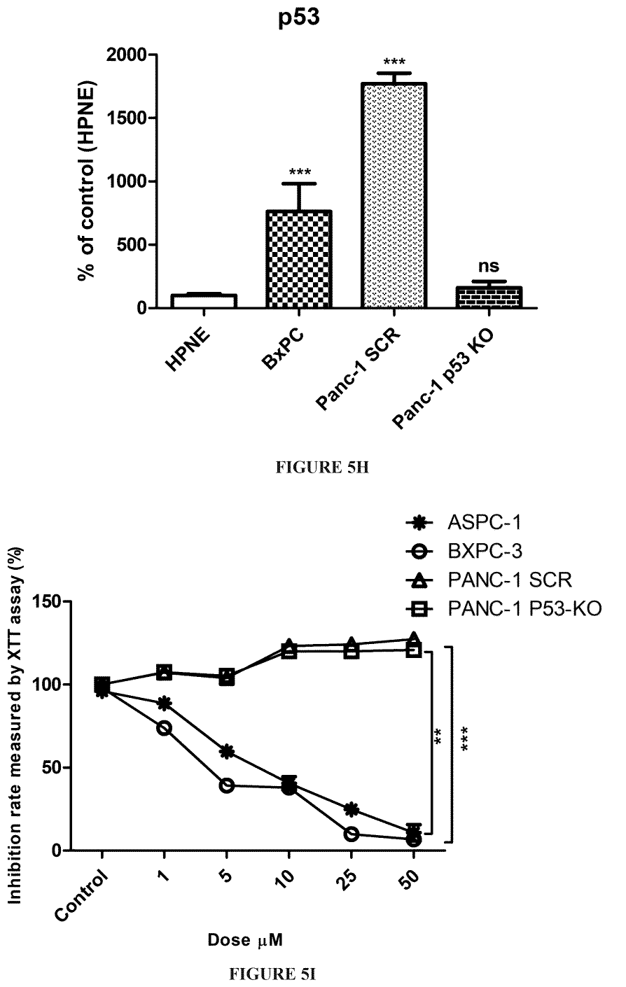

To further investigate the biological mechanism of Eravacycline's inhibition of BxPC-3 cells growth, the expression of p53 and POLK-related protein, two of the top-ranked targets obtained with the in-silico yeast screening model, was examined by performing western blot assays. The effects of increasing concentrations of eravacycline for 72 h on the expression of the p53 and POLK gene in BxPC-3 cells was examined. It was found that the protein expression of POLK and p53 was significantly reduced after treatment with Eravacycline in a dose-dependent manner as compared to the control group (FIGS. 5A-5B, and 5E-5F). Neither gemcitabine nor doxorubicin reduced p53 and POLK protein expression, indicating that eravacycline induces apoptosis by a different mechanism than these chemotherapeutics. It was also found that baseline POLK protein expression was significantly higher in pancreatic cancer cells (BxPC-3 cell line) as compared to normal pancreatic cells (HPNE cell line) (FIGS. 5C-5D). This may partially explain the increased effect of eravacycline on pancreatic cancer cells verses healthy pancreatic cells (FIG. 2E).

### Example 7

### Eravacycline Suppresses the Survival of PDAC Cells by Inhibiting Mutant p53 Gene Expression

TP53 (encoding the p53 protein) plays a central role in tumor suppression. TP53 tumor suppressor gene is the most frequently mutated gene in human cancer, including more than 50% of pancreatic adenocarcinomas. Even though p53 is the most frequently mutated gene in human cancer, and over half of human cancers contain p53 mutations, the majority of p53 mutations in cancer are missense mutations, leading to the expression of full-length mutant p53 protein. While the critical role of wild-type p53 in tumor suppression has been firmly established, increasing evidence indicates that many tumor-associated mutant p53 proteins lose their wild-type p53 tumor suppressor function, and acquire new activities that contribute to tumor development, suggesting an oncogenic role of mutant p53 by gain-of-function mechanisms. The mutant p53 protein is often present in high levels in tumors, contributing to malignant progression. In recent years, scientists have identified the mutant p53 gene as an attractive therapeutic target for cancer. Many mutant p53 gains of function have been identified, including those that promote tumor cell proliferation, survival, migration, and invasion, improve chemoresistance, disrupt normal tissue architecture, and promote cancer metabolism. Previous reports also showed that mutant P53 gain of function, demonstrated in P53 knock-in mouse models, develop more aggressive tumors or earlier tumors compared with p53 null mice.

Testing the effect of eravacycline on the expression of the TP53 gene, the expression of p53, which is mutated in the BxPC-3 cell line, was found to be significantly reduced at the protein level after treatment with eravacycline in a dose and time-dependent manner (FIGS. 5E-5F).

Mutant p53 protein was found to be present in high levels in pancreatic cancer cell lines (Bxpc-3 and Panc-1 scr) as compared to normal pancreatic cell line hTERT-HPNE (FIGS. 5G-5H). This observation is consistent with previous reports which also showed that the mutant p53 protein is often present in high levels in tumors. This tumor accumulation is critical for mutant p53 to exert its gain of function in tumorigenesis and contributes to more advanced tumors. Additionally, these data indicated that mutant P53 protein reduction might play a potent role in eravacycline-induced inhibition of cancer cell proliferation.

### Example 8

### The Effect of Eravacycline on Cell Viability of Different Types of Pancreatic Cancer p53 Mutations Status

To further investigate the effect of eravacycline on the cancer cell viability of different types of pancreatic cancer p53 mutations, human PDAC (AsPC-1, BxPC-3, Panc-1 scr, Panc-1 p53-R273H knock-out (KO)) cell lines were treated with varying concentrations of eravacycline (1, 5, 10, 25, and 50 μM), with a medium used as a control, for 72 h. Then, an XTT assay was performed to determine the growth inhibition rate (FIG. 5I). After 72 hours of treatment, eravacycline was shown to significantly decrease the BxPC3-3 cells' growth by 90% (P<0.01) and 93% (P<0.001) at 25 and 50 μM concentrations, respectively. In contrast, it caused a 75% (P<0.05) and 89% (P<0.001) decrease in AsPC-1 cell growth at the same concentrations (FIG. 5I). No significant effect was observed for eravacycline in the Panc-1 cell lines (FIG. 5I). Eravacycline shows strong and selective viability reduction in the p53-Y220C cancer cell line BXPC-3, while no toxicity was observed in the same concentration range in the p53-R273H mutant cell line Panc-1. Without being bound to any particular theory, this suggests that eravacycline restored p53 function. Possibly, binding of eravacycline stabilizes the structure of p53 protein which affects its DNA binding and/or thermodynamic stability and reactivates apoptotic signaling pathways in tumor cells either by transactivation-dependent or independent pathways. Or, alternatively, by blocking the MDM2-p53 interaction, wild-type p53 function is retained and its tumor suppressor function is reactivated.

### Example 9

### The Effect of Eravacycline on the Growth of PDAC Xenografts

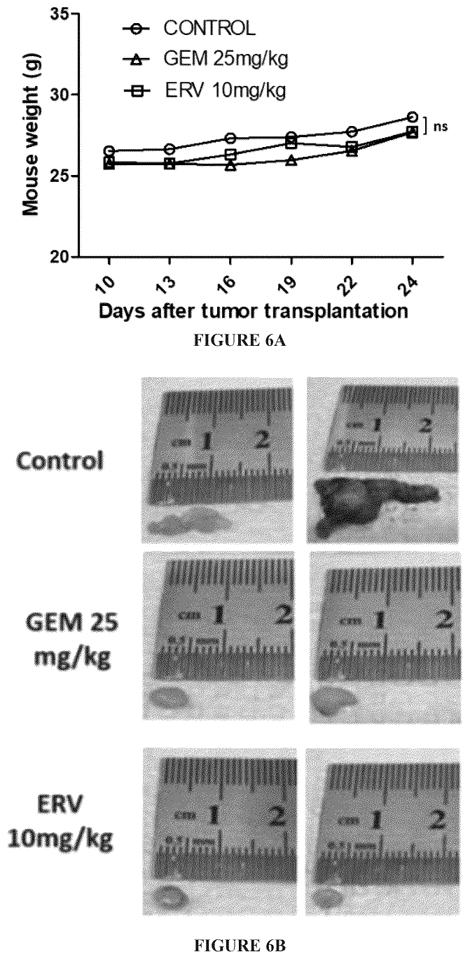

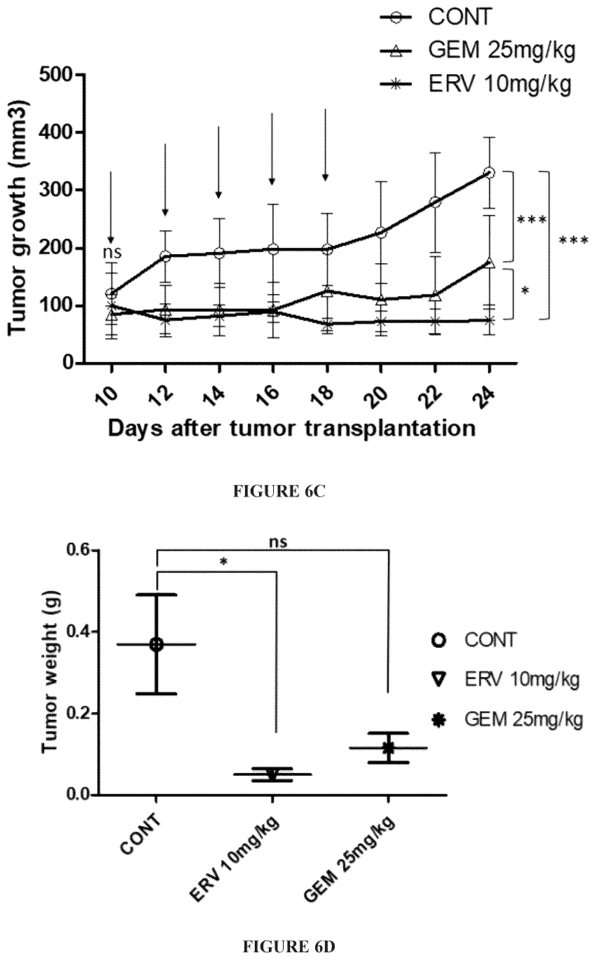

To further assess the inhibition effect of eravacycline on human PDAC cells in vivo, BxPC-3 cells were injected subcutaneously into nude mice to establish xenograft models for in vivo experiments. The mice were randomly divided into three groups: the control group, the gemcitabine group (25 mg/kg), and the eravacycline group (10 mg/kg). Following the last administration of treatment, mice were monitored for an additional six days; then the experiment was terminated, the mice were sacrificed, and the tumors were excised, photographed, and weighed (FIGS. 6B-6D). The results revealed that eravacycline treatment significantly blocked tumor growth by 76%, (FIG. 6C). However, there was no significant weight loss overall in the mice in the eravacycline group, compared with the control group (FIG. 6A). Further, eravacycline treatment was found to be superior to gemcitabine treatment both as evaluated by tumor volume (FIG. 6C) and tumor weight (FIG. 6D). These data show that eravacycline effectively inhibited the development of tumors in vivo and in a manner that was even superior to gemcitabine.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A method of treating or preventing pancreatic cancer or a metastasis thereof in a subject in need thereof, the method comprising administering to said subject a therapeutically effective amount of eravacycline or a derivative thereof, thereby treating or preventing pancreatic cancer or a metastasis thereof in the subject.

2. A method of treating or preventing a cancer comprising expression of a mutated p53 bearing a mutation wherein Tyrosine in position 220 is substituted by Cysteine (Y220C) in a subject in need thereof, the method comprising administering to said subject a therapeutically effective amount of eravacycline or a derivative thereof, thereby treating or preventing cancer comprising expression of a mutated p53 bearing a Y220C mutation in the subject.

3. The method of claim 2, wherein said cancer is pancreatic cancer or a metastasis thereof.

4. The method of claim 1, wherein said eravacycline is represented by formula I:

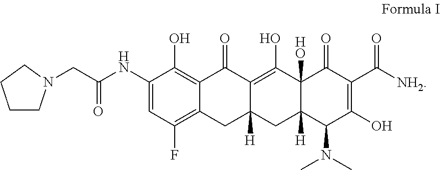 [SMILES: CN(C)[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12]

5. The method of claim 1, wherein said eravacycline is a salt or crystalline form of eravacycline.

6. The method of claim 5, wherein said eravacycline is eravacycline dihydrochloride.

7. The method of claim 1, wherein said pancreatic cancer is pancreatic ductal adenocarcinoma (PDAC).

8. The method of claim 1, wherein said administering is systemic administering.

9. The method of claim 1, wherein said administering comprises administering a pharmaceutical composition comprising a therapeutically effective amount of said eravacycline and a pharmaceutically acceptable carrier, excipient or adjuvant.

10. The method of claim 1, wherein said subject does not suffer from a bacterial infection treatable with eravacycline.

11. The method of claim 1, wherein said treating comprises at least one of: increasing cancer cell apoptosis, increasing expression of cleaved poly(ADP-ribose) polymerase 1 (cPARP1) in a cancer cell, decreasing expression of DNA polymerase kappa (POLK) in a cancer cell, decreasing expression of mutated p53 in a cancer cell, and decreasing migration of a cancer cell, in the subject.

12. The method of claim 1, further comprising administering at least one other conventional cancer therapy.

13. A kit comprising a pharmaceutical composition comprising a therapeutically effective amount of eravacycline or a derivative thereof and a conventional cancer therapy.

14. (canceled)

15. (canceled)

16. The kit of claim 13, wherein said eravacycline is represented by formula I:

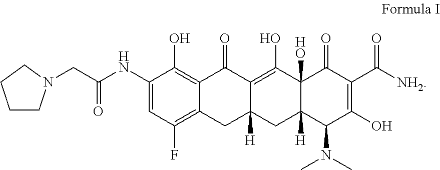 [SMILES: CN(C)[C@@H]1C(O)=C(C(N)=O)C(=O)[C@@]2(O)C(O)=C3C(=O)c4c(O)c(NC(=O)CN5CCCC5)cc(F)c4C[C@H]3C[C@@H]12]

17. The kit of claim 13, wherein said eravacycline is a salt or crystalline form of eravacycline.

18. The kit of claim 17, wherein said eravacycline is eravacycline dihydrochloride.

19. (canceled)

20. The kit of claim 13, wherein said pharmaceutical composition is formulated for systemic administration.

21. The kit of claim 13, wherein said pharmaceutical composition further comprises a pharmaceutically acceptable carrier, excipient, or adjuvant.

22. (canceled)

23. (canceled)

24. (canceled)

25. The kit of claim 13, wherein said conventional cancer therapy is selected from a chemotherapy, an immunotherapy, and a targeted therapy.
